(19) United States
(12) Patent Application Publication
(10) Pub. No.: US 2023/0405090 A1
(43) Pub. Date: Dec. 21, 2023

(54) ANTIVIRAL AGENTS

(71) Applicant: Quantec Limited, Hamilton (NZ)

(72) Inventors: Rodney Wayne CLAYCOMB, Hamilton (NZ); Colin Roger OGLE, Hamilton (NZ); Katharine Helen ADAM, Hamilton (NZ)

(73) Assignee: Quantec Limited, Hamilton (NZ)

(21) Appl. No.: 18/252,009

(22) PCT Filed: Nov. 4, 2021
(86) PCT No.: PCT/NZ2021/050195

§ 371 Date: May 5, 2023

(30) Foreign Application Priority Data
Nov. 6, 2020 (NZ) 769680
Dec. 22, 2020 (NZ) 771493
Mar. 23, 2021 (NZ) 774216

(51) Int. Cl. A61K 38/40; A61K 45/06; A61P 31/16; A61K 38/18; A61K 38/44; C07K 16/10; A61K 38/47; A61K 38/46

(52) U.S. Cl. CPC A61K 38/40; A61K 45/06; A61P 31/16; A61K 38/1891; A61K 38/44; C12Y 108/03002; A61K 38/47; A61K 38/465; C12Y 111/01017; C12Y 302/01024; C07K 16/10

(57) ABSTRACT

The present invention relates to products and processes for the treatment or prevention of viral infection(s). In particular the invention relates to the use of one or more proteins, typically obtained from milk, for the treatment or prevention of viral infection(s). In particular embodiments the invention uses combinations of milk proteins for the treatment or prevention of viral infection(s). The products of the invention may be used in combination with other active agents, including other antiviral agents.

### FIELD OF INVENTION

The present invention relates to products and processes for the treatment or prevention of viral infection(s). In particular the invention relates to the use of one or more proteins, typically obtained from milk, for the treatment or prevention of viral infection(s). In particular embodiments the invention uses combinations of milk proteins for the treatment or prevention of viral infection(s). The products of the invention may be used in combination with other active agents, including other antiviral agents.

### BACKGROUND OF THE INVENTION

A virus is a small infectious agent that replicates only inside the living cells of an organism. Examples of conditions caused by viral pathogens include the common cold, influenza, chickenpox, and cold sores.

The natural immune response of an animal infected with a virus may be sufficient to ameliorate the effects of, or even eliminate the infecting virus. Immune responses can also be accelerated by the use of vaccines, which can even confer practical immunity against a specific viral infection. The specificity of vaccines is advantageous in that the vaccine can stimulate a strong response, and also disadvantageous in that they may not be effective against even closely related viruses.

Furthermore, vaccines do not exist for all viruses, and with over 200 known viruses responsible for seasonal colds and flus it is not practical to inoculate for all of them. Young children experience an average of 8 to 10 colds a year.

In addition to prophylactic vaccine treatments, numerous antiviral agents have been developed which are typically used to prevent, inhibit, or reduce the viral activity of a virus on or in a subject. Part of the challenge in developing antiviral agents is that intrinsically the virus relies on a host organism's cells to replicate. As such, it can be difficult to successfully locate a target for the antiviral agents that is effective against the virus without adversely affecting the host organism's cells.

Cold sores are caused by herpes simplex virus (HSV-1), and are a common occurrence. Once infected a person may experience recurrence of the sores. Antiviral medication can reduce the severity of symptoms and reduce the occurrences, but are available on prescription only. For most cases over the counter topical treatments can only provide relief from the symptoms, and do not address the underlying cause of the symptoms. HSV-2 can cause genital herpes.

The symptoms of a viral infection can vary from mild to severely debilitating. If left untreated, viral infections can cause death. Numerous viruses, including those that cause AIDS, HPV infection, and viral hepatitis, can result in chronic infections.

Human Influenza can lead to high fever, runny nose, sore throat, muscle and joint pain, headache, coughing, feeling tired, and death in a significant number of cases. Each year Human Influenza results in about three to five million cases of severe illness and about 290,000 to 650,000 deaths. Of the known genera of influenza viruses (including influenza virus A, B, C, D), influenza virus A has led to a significant number of pandemics that have killed millions of people. The influenza virus A can be subdivided into different serotypes based on the antibody response to these viruses, including:
H1N1, which caused Spanish flu in 1918, and Swine Flu in 2009
H2N2, which caused Asian Flu in 1957
H3N2, which caused Hong Kong Flu in 1968
H5N1, which caused Bird Flu in 2004
H7N7, which has unusual zoonotic potential
H1N2, endemic in humans, pigs and birds
H9N2
H7N2
H7N3
H10N7
H7N9, rated in 2018 as having the greatest pandemic potential among the Type A subtypes
H6N1, which only infected one person, who recovered.

Two classes of antiviral medications have been discovered—the neuraminidase inhibitors and the M2 inhibitors, however the efficacy of these medications is not strong, and resistance has developed against both classes.

Coronaviruses are enveloped RNA viruses that infect mammals and birds. The severe acute respiratory syndrome (SARS) and the Middle East respiratory syndrome (MERS) are both members of the genus Betacoronavirus, and responsible for hundreds of deaths in Asia and the Middle East, respectively. The late 2019 emergence of the novel, SARS-coronavirus 2 (SARS-CoV-2) pathogen, with rapid human to human transmission and international spread, poses an immediate global health emergency. In response, a global effort for effective treatments is underway following the World Health Organisation's (WHO) declaration of a pandemic, based on the substantial number of cases of the SARS-CoV-2 illness (COVID-19) globally. There is an urgent need for both an effective coronavirus vaccine to prevent the spread of this virus and in parallel, novel therapeutic strategies to reduce the global mortality numbers.

Identifying therapeutic strategies is considered to be the fastest means of addressing this pandemic. However, due to unintended side effects, in addition to a lack of substantial evidence to demonstrate their efficacy in treating coronavirus infection, there is still a clear unmet clinical need to develop new treatment options specifically for coronavirus.

Per the Baltimore classification, coronarvisues belong to the family Coronaviridae which includes four genera, being the alphacoronavirus, betacoronavirus (β-CoVs), gammacoronavirus, and deltacoronavirus. The alphacoronaviruses and betacoronaviruses infect a wide range of species, including humans. In this regard, the β-CoVs that are of particular clinical importance in humans include OC43 and HKU1 of the A lineage, Severe Acute Respiratory Syndrome coronavirus (SARS-CoV) and SARS-CoV-2 (which causes the disease COVID-19) of the B lineage, and Middle Eastern Respiratory Syndrome-related coronavirus (MERS-CoV) of the C lineage. As new viral strains emerge, there continues to be a need to develop new, safe, and effective antiviral therapies. Ideally, antiviral therapies may be available as health and wellness products that have direct antiviral properties and/or support the immune system to respond effectively to exposure.

The present invention aims to address one or more of the foregoing problems or at least provide the public with a useful choice.

### SUMMARY OF THE INVENTION

In a first aspect the invention provides a method of preventing, inhibiting, or reducing the viral activity of a virus on or in a cell, the method including the step of contacting the virus or the cell with an effective amount of a combination of at least two proteins, each protein having an isoelectric point of or above substantially 6.8 and which are extracted from milk.

In a second aspect the invention provides a method of preventing, inhibiting, or reducing the viral activity of a virus on or in a subject, the method including the step of administering to the subject an effective amount of a combination of at least two proteins, each protein having an isoelectric point of or above substantially 6.8 and which are extracted from milk.

In a third aspect the invention provides a method of treating or preventing a viral infection in a subject, the method including the step of administering to the subject an effective amount of a combination of at least two proteins, each protein having an isoelectric point of or above substantially 6.8 and which are extracted from milk.

In a fourth aspect the invention provides the use of a combination of at least two proteins, each protein having an isoelectric point of or above substantially 6.8 and which are extracted from milk, in the manufacture of a medicament for the prevention, inhibition or reduction of the viral activity of a virus on or in a cell.

In a fifth aspect the invention provides the use of a combination of at least two proteins, each protein having an isoelectric point of or above substantially 6.8 and which are extracted from milk, in the manufacture of a medicament for the prevention, inhibition or reduction of the viral activity of a virus on or in a subject.

In a sixth aspect the invention provides the use of a combination of at least two proteins, each protein having an isoelectric point of or above substantially 6.8 and which are extracted from milk, in the manufacture of a medicament for the treatment or prevention of a viral infection in a subject.

In a seventh aspect the invention provides a combination of at least two proteins, each protein having an isoelectric point of or above substantially 6.8 and which are extracted from milk, for the prevention, inhibition or reduction of the viral activity of a virus on or in a cell.

In an eighth aspect the invention provides a combination of at least two proteins, each protein having an isoelectric point of or above substantially 6.8 and which are extracted from milk, for the prevention, inhibition or reduction of the viral activity of a virus on or in a subject.

In a ninth aspect the invention provides a combination of at least two proteins, each protein having an isoelectric point of or above substantially 6.8 and which are extracted from milk, for the treatment or prevention of a viral infection in a subject.

It has now been found that a combination of at least two proteins, each protein having an isoelectric point of or above substantially 6.8 and which are extracted from milk, has antiviral activity.

In preferred embodiments the virus(es) against which the combination displays antiviral activity is:
i. a strain from the family Coronaviridae; such as a strain from the subfamily Orthocoronavirinae; such as a strain from one of the genera alphacoronavirus, betacoronavirus (β-CoVs), gammacoronavirus, and deltacoronavirus; such as a strain from the genus betacoronavirus; such as a SARS virus; such as a SARS-CoV strain and/or a SARS-CoV-2 strain; such as COVID-19;
ii. a Human Influenza virus, such as a Human Influenza A virus, such as Human Influenza A H1N1; and/or
iii. a Herpes Simplex virus, such as HSV-1 or HSV-2, such as HSV-1.

The products and processes of the invention may be used in the treatment or prevention of a viral infection in any one of a number of subjects. For example, the products and processes of the invention may be used in the treatment or prevention of a viral infection in a mammal, such as a domesticated animal (such as a cow, sheep, horse, cat, dog, goat, rabbit) or in a human. Typically the products and processes of the invention are formulated for the treatment or prevention of a viral infection in a human.

The at least two proteins in the combination may be used separately, simultaneously, or sequentially. The at least two proteins in the combination will generally be provided in intimate admixture, namely in a composition.

One approach to preventing, inhibiting, or reducing the viral activity of a virus on or in a cell or subject, and/or treating or preventing a viral infection in a subject, is to use a combination of at least two antiviral agents, typically having different mechanisms of action. This approach has been partially successful in treating subjects infected by HIV, for example. As such the present invention also provides methods and uses of the combination of at least two proteins, each protein having an isoelectric point of or above substantially 6.8 and which are extracted from milk, in further combination with one or more antiviral agents. Examples of such known antiviral agents that may be used in combination with the at least two proteins, each protein having an isoelectric point of or above substantially 6.8 and which are extracted from milk, include (together with an example of their intended viral target):

Abacavir (HIV); Acyclovir (Aciclovir) (Herpes Simplex); Adefovir (Hepatitis B); Amantadine (Influenza); Ampligen (Avian Influenza); Amprenavir (Agenerase) (HIV); Umifenovir (Arbidol) (Influenza); Atazanavir (HIV); Atripla (HIV); Baloxavir marboxil (Xofluza) (Influenza A, Influenza B); Biktarvy (HIV); Boceprevir (Hepatitis C); Bulevirtide (Hepatitis D and Hepatitis B); Cidofovir (AIDS); Cobicistat (Tybost) (HIV); Combivir (HIV); Daclatasvir (Daklinza) (Hepatitis C); Darunavir (HIV); Delavirdine (Hepatitis C); Descovy (Hepatitis B); Didanosine (HIV); Docosanol (Herpes Simplex); Dolutegravir (HIV); Doravirine (Pifeltro) (HIV); Edoxudine (Herpes Simplex); Efavirenz (HIV); Elvitegravir (HIV); Emtricitabine (HIV); Enfuvirtide (HIV); Entecavir (HIV); Etravirine (Intelence) (HIV); Famciclovir (Herpes Zoster); Fomivirsen (AIDS); Fosamprenavir (HIV); Foscarnet (Herpes); Ganciclovir (Cytovene) (Cytomegalovirus (CMV)); Ibacitabine (Herpes labialis); Ibalizumab (Trogarzo) (HIV); Idoxuridine (Herpes); Imiquimod (Genital wart); Imunovir (Herpes Simplex); Indinavir (HIV); Lamivudine (HIV); Letermovir (Prevymis) (Cytomegalovirus (CMV)); Lopinavir (HIV); Loviride (HIV); Maraviroc (HIV); Methisazone (Smallpox); Moroxydine (Influenza); Nelfinavir (HIV); Nevirapine (HIV); Nexavir (formerly Kutapressin) (Herpes Zoster); Nitazoxanide (Broad-spectrum antiviral); Norvir (HIV); Oseltamivir (Tamiflu) (Influenza); Penciclovir (Herpes); Peramivir (Influenza); Penciclovir (Herpes); Peramivir (Rapivab) (Influenza); Pleconaril (Picornavirus); Podophyllotoxin (Genital wart); Raltegravir (HIV); Remdesivir (COVID-19); Ribavirin (Hepatitis C); Rilpivirine (HIV); Rimantadine (Influenza A); Ritonavir (HIV); Saquinavir (HIV); Simeprevir (Olysio) (Hepatitis C); Sofosbuvir (Hepatitis C); Stavudine (HIV); Taribavirin (Viramidine) (Hepatitis Syndromes in which Ribavirin is active); Telaprevir (Hepatitis C); Telbivudine (Tyzeka) (Hepatitis B); Tenofovir alafenamide (Hepatitis B); Tenofovir disoproxil (Hepatitis B, HIV); Tipranavir (HIV); Trifluridine (Eye related Herpes); Trizivir (HIV); Tromantadine (Herpes Simplex); Truvada (HIV); Umifenovir (Influenza); Valaciclovir (Valtrex) (Herpes Simplex, Herpes Zoster); Valganciclovir (Valcyte) (HIV); Vicriviroc (HIV-1); Vidarabine (Herpes Simplex, Varicella Zoster); Zalcitabine (HIV); Zanamivir (Relenza) (Influenza A, Influenza B); Zidovudine (HIV).

In a tenth aspect the invention provides a combination of:
(i) a combination of at least two proteins, each protein having an isoelectric point of or above substantially 6.8 and which are extracted from milk; and
(ii) at least one antiviral agent.

The ‘at least one antiviral agent’ may be selected from either the formulation or the active pharmaceutical ingredient (API) in any of the aforementioned known antiviral agents.

In an eleventh aspect the invention provides a combination of at least two proteins, each protein having an isoelectric point of or above substantially 6.8 and which are extracted from milk, wherein where the combination includes lactoferrin the lactoferrin content of the combination is less than 40% w/w.

The inventors have discovered that milk protein combinations having low lactoferrin contents may be surprisingly potent against certain viruses, including Human Influenza (such as Human Influenza A); a virus of the family Coronaviridae (such as COVID-19); and/or a Herpes Simplex virus (such as HSV-1). In some embodiments the invention provides a combination of at least two proteins, each protein having an isoelectric point of or above substantially 6.8 and which are extracted from milk, wherein where the combination includes lactoferrin the lactoferrin content of the combination is less than 30% w/w, such as less than 20% w/w, such as less than 10% w/w. Such combinations may be present as a composition such that where the composition includes lactoferrin the lactoferrin content of the composition is less than 40% w/w, such as less than 30% w/w, such as less than 20% w/w, such as less than 10% w/w.

Further aspects of the invention, which should be considered in all its novel aspects, will become apparent to those skilled in the art upon reading of the following description.

### BRIEF DESCRIPTION OF THE DRAWINGS

One or more embodiments of the invention will be described below by way of example only, and without intending to be limiting, with reference to the following drawings, in which:

FIG. 1 shows a dose-inhibition curve for test sample #3 against Human Influenza H1N1 A, demonstrating an IC₅₀ of 193.6 and no cytotoxicity;

FIG. 2 shows a dose-inhibition curve for test sample #4 against Human Influenza H1N1 A, demonstrating an IC₅₀ of 93.91 and extrapolated cytotoxicity;

FIG. 3 shows a dose-inhibition curve for lactoferrin (Lf) against Human Influenza H1N1 A, demonstrating an IC₅₀ of 272.4 and no cytotoxicity;

FIG. 4 shows a dose-inhibition curve for combination #4 against rVSV-SARS-CoV-2 S, demonstrating an IC₅₀ of 70.04 and extrapolated cytotoxicity;

FIG. 5 shows a dose-inhibition curve for combination #6 against rVSV-SARS-CoV-2 S, demonstrating an IC₅₀ of 89.68 and extrapolated cytotoxicity.

FIG. 6 shows a dose-inhibition curve for freeze-dried lactoferrin (Lf) against rVSV-SARS-CoV-2 S, demonstrating an ICso of 79.75 and extrapolated cytotoxicity; and

FIG. 7 shows a dose-inhibition curve for spray-dried lactoferrin (Lf) against rVSV-SARS-CoV-2 S, demonstrating an IC₅₀ of 56.64 and extrapolated cytotoxicity.

### DETAILED DESCRIPTION OF THE INVENTION

Preferred features of the combination of proteins (for example the cationic fraction of milk)

Throughout this specification, use of the term ‘cationic fraction’ should be taken as meaning a fraction or isolated components from a milk, being cationic components that bind to cation exchange media, and include any component of milk which has an isoelectric point of or above substantially 6.8.

Without wishing to be bound by theory, the inventors believe that some or all the proteins in the cationic fraction isolated from milk are collectively working together as antiviral agents. It is believed that two or more of the proteins are acting synergistically together as antiviral agents.

Without wishing to be bound by theory, the combination of at least two proteins may provide a single, or plural, mode of action. For example, the proteins in the combination could independently or in combination be inhibiting a part or whole of the virus itself (e.g., a spike protein) and/or interacting with the cell itself to prevent viral adhesion and/or entry.

As used herein, the term “synergistic” (and variants thereof) means that the effect achieved with the compositions and combinations of the invention is greater than the sum of the effects that result from using the individual components as a monotherapy. Advantageously, such synergy provides greater efficacy at the same doses, and provides an effect where otherwise there would be no discernible effect.

It should be understood that the particular method of combining the proteins in the composition together, which appear to provide advantageous selectivity, should not be considered to be a limitation to the invention at hand. For instance, a person skilled in the art could potentially prepare a combination of proteins from different sources, or even potentially synthetically engineer each protein and combine them as appropriate. However, the ability to separate and elute a cationic fraction of proteins from a milk sample using chromatographic methods represents a convenient way to prepare the combination(s) of the invention as a composition, and also provides a delicate mechanism to keep the proteins in their innate environment to avoid loss of protein function or inter-engagement with the other milk proteins, and to promote any form of synergism that appears to be at play between the proteins. As such fractionation of milk is a preferred method of forming the combination of milk proteins of the invention.

The proteins used in the combination, such as the composition, may be isolated or extracted from one or more sources of milk, such as bovine milk, sheep milk, goat milk, buffalo milk, camel milk, human milk and the like. While bovine milk is preferred, the major and minor proteins found in bovine milk are also found in other sources of milk, with very similar isoelectric points in each case. Additionally, the term milk should be taken to include whole milk, skim milk or whey.

In one preferred embodiment the cationic fraction may have a molecular weight distribution of 3,000-80,000 Daltons by SDS-PAGE. This protein size distribution range encompasses the size of the proteins observed within the cationic fractions (and sub-fractions) of milk.

The most prevalent proteins in the combination of proteins of the invention are lactoferrin, angiogenin and lactoperoxidase. The relative amounts of these proteins can vary in milk. In some embodiments, the combination of milk proteins used in the invention may include:
lactoferrin in a range between about 0% w/w and about 90% w/w; such as between about 1% w/w and about 70% w/w; such as in a range between about 5% w/w and about 70% w/w; and/or
lactoperoxidase in the range between about 0% w/w and about 80% w/w; such as between about 1% w/w and about 80% w/w; such as in a range between about 1% w/w and about 70% w/w; and/or
angiogenin in the range of 0-30% w/w, such as in a range between about 0% w/w and about 15% w/w; such as in a range between about 1% w/w and about 10% w/w.

The combination of milk proteins used in the invention may be combined with other components that are not proteins having an isoelectric point of or above substantially 6.8 and which are extracted from milk. The exemplary ranges provided herein for milk protein concentrations in the combination of the invention should be interpreted as being proportions of either the total combination used in the invention, or the total milk protein fraction of the combination. For example, where lactoferrin is stated as being about 45% w/w, this reference includes disclosure of:
a composition in which lactoferrin is 45% weight per weight of the entire composition; or
alternatively 45% weight per weight of the protein fraction of the composition; or
alternatively 45% weight per weight of those proteins in the composition which have an isoelectric point of or above substantially 6.8.

Without wishing to be bound by theory it is believed that combinations of milk proteins wherein the lactoferrin content is relatively low may provide enhanced activity against certain viruses, such as human influenza, such as human influenza A type, such as human influenza A type H1N1. For example, the lactoferrin content of the milk protein combination may be less than 30% w/w, such as less than 20% w/w, such as less than 10% w/w. This result was particularly surprising since it had been thought that lactoferrin may be contributing to the antiviral activity of the milk protein to a significant, if not major extent. This result suggests that other component(s) of the milk protein combination are more potent antiviral agents (alone or in combination) than lactoferrin.

There are a wide number of additional proteins in milk which may be isolated as part of the cationic fractions and combinations, such as a composition, studied by the inventors, many of which may also be contributing towards the antiviral activity.

Without limitation, the proteins found in the cationic fraction of milk, and also considered to be relevant to the invention at hand and may be considered separately to be variously preferable to include in the combination of the invention, are discussed in more detail below. It should be appreciated that although lactoferrin itself has been previously shown to have some antiviral activity against some viruses, many of the other proteins in milk have not previously been shown to have any antiviral activity at all.

### Lactoperoxidase

Lactoperoxidase (Lp) is a protein present in the mammary gland secretion and many other exocrine secretions of mammals.

U.S. Pat. No. 6,544,498 (the entire contents of which are incorporated herein by reference) discloses the extraction by gradient elution of a basic protein fraction which has an isoelectric point between 7.5 and 11 and a molecular weight distribution of 3,000 to 80,000 Daltons, with the main components being lactoperoxidase and lactoferrin.

### Lactoferrin

Lactoferrin (Lf) is a glycoprotein which is present in mammary gland secretion and many other exocrine secretions of mammals. Lf is secreted predominately by surface epithelia into the mucosal environment. Lactoferrin is a multifunctional protein that has antibacterial, antifungal, antiviral, antitumour, anti-inflammatory, and immunoregulatory properties.

Lf is produced at high levels in nasal and tracheal passages, and in gastric, genital and ophthalmic secretions. Lf is also produced at high levels in neutrophils where it is stored in secondary granules and released during inflammation.

The highly basic N terminal region of bovine lactoferrin is thought to be essential for antimicrobial activity. The 25 N-terminal amino acids may be removed by proteases to form lactoferricin (Lfcin). These proteases may be naturally occurring in milk or serum, and many micro-organisms produce proteases. Lfcin is up to a 1000 fold more effective against some micro-organisms than intact lactoferrin. Lfcin has been shown to inhibit a diverse range of microorganisms such as gram-negative bacteria, gram-positive bacteria, yeast, filamentous fungi, and parasitic protozoa, including some antibiotic-resistant pathogens. The present invention contemplates that lactoferricin may be added to the combination, such as the composition, replace lactoferrin, and/or be a natural degradation product of lactoferrin in the combination of the present invention due to proteolytic action.

Current commercial applications of bovine Lf include infant formulas, fermented milks, nutritional iron supplements, chewing gums, immune-enhancing nutraceuticals, cosmetic formulas and feed and pet care supplements. Therefore, it is advantageous to note that there is general consumer acceptance, and food safety regulations for use of Lactoferrin in the combination, such as the composition.

### Angiogenin

Angiogenin belongs to the ribonuclease superfamily which have been identified in milk.

Lysozyme-Like Proteins, such as Chitinase-Like Protein (CLP-1) or Lysosomal Alpha Mannosidase (LAM)

The combination of the invention may include lysozyme-like protein, such as chitinase-like protein (CLP-1) or lysosomal alpha mannosidase (LAM). Lysozyme-like proteins (such as CLP-1 or LAM) have cell lysing activity.

The combination (such as the cationic fraction) may include quiescin and/or jacalin-like protein.

Other milk proteins that may individually be considered preferable to include within the combination to improve its effectiveness (either through imparting selectivity, or some other form of indirectly modulation of the protein(s) functionality) include:
cathelicidin 1, 3 and/or 6;
N-acetyl glucosaminidase;
serum amyloid A;
Defensin;
Peptidoglycan recognition protein;
Xanthine dehydrogenase;
Immunoglobulin(s) IgA, IgD, IgG, IgM, IgA, and/or IgE;
Growth factors EGF, IGF 1, TGF B1 and TGF B2.

Immunoglobulins are important components of milk as a food source as they provide passive protection to the suckling young. Although they are not strongly cationic some immunoglobulins, IgG, IgM, IgA and polymeric immunoglobulin receptor (PIGR) can be extracted by cation exchange methodologies.

It is anticipated that the combination (such as the cationic fraction) isolated from milk may also include small amounts of a number of growth factors; although these growth factors may be present at low levels, their action can be potent in stimulating cell repair. These growth factors may include for example: EGF, IGF 1, TGF B1 and TGF B2.

Smolenski et al. (2007) reported on the identity and significant number of minor proteins in bovine milk by Mass Spectrometry (MS) and, in particular, identified a significant number of milk proteins that are involved in host defense. The results are shown in Table 1, which have been adapted to show, in bold, some of the proteins which correspond to those which may be preferably incorporated into the combination of the present invention (and which were isolated via the cationic fraction in milk and shown to have high selectivity according to the present invention). It should be noted that Smolenski et al. (2007) used SDS-PAGE methods that do not disclose the detection of the proteins identified in the combinations (such as compositions including the cationic fraction) used in the present invention (e.g. angiogenin, jacalin-like protein, quiescin, PIGR and the growth factors).

Table 1. Host defense-related minor proteins identified from milk, showing some of those that may be extracted as part of the cationic fraction (bold) (reproduced from Smolensk' et al., 2007)

TABLE 1
Minor proteins identified in bovine milk.

| ACC Number | Protein Name | Function | pl |
|---|---|---|---|
| NP_777250 | cathelicidin 1 (Bactenecin | antimicrobial properties | 6.8* |
| | 1) | | |
| AAB64304 | chitinase-like protein 1 | eosinophil chemotactic properties | 8.8 |
| | (CLP-1) | | |
| Q290092 | endoplasmin precursor | participates in the assembly of | 4.7 |
| | (GRP94/GP96) | antibody molecules and signaling | |
| | | molecule for polymorphonuclear | |
| | | neutrophils | |
| NP_776758 | glucose regulated protein | regulates signaling by interacting with | unknown |
| | 58 kDa | stat3 | |
| NP_776770 | heat shock 70 kDa protein 8 | activated through proinflammatory | 5.4 |
| | | response mechanisms enhancing MMP- | |
| | | 9 expression in monocytic cells | |
| NP_071705 | heat shock 70 kDa protein 5 | upregulation in macrophages upon IL-4 | unknown |
| | (glucose-regulated protein) | stimulation | |
| AAA18337 | heat shock protein 27 | inhibitor of neutrophil apoptosis | 5.98* |
| BAA32525 | heat shock protein 70 kDa | stress response (refolding and | 5.68* |
| | protein 1A | degradation of denatured proteins) | |
| AAC98391 | immunoglobulin IgA | antigen recognition | [symbol] |
| AAN07166 | immunoglobulin IgD | antigen recognition | [symbol] |
| AAB37381 | immunoglobulin IgG | antigen recognition | [symbol] |
| AAN60017 | immunoglobulin IgM | antigen recognition | [symbol] |
| AAQ88452 | IRTA2 | B-cell immunoglobulin super-family | unknown |
| | | receptor | |
| AAA30617 | lactoferrin | iron binding and antimicrobial peptide | 8.67* |
| | | ″lactoferricin″ | |
| NP_776358 | lactoperoxidase | oxidative peroxidase activity | 8.327* |
| BAA07085 | lymphocyte cytosolic | regulation of neutrophil integrin | 5.21* |
| | protein 1 (65K macrophage | function | |
| | protein/L-plastin) | | |
| P21758 | macrophage scavenger | mediate the binding, internalization | 5.7* |
| | | and processing of negatively charged | |
| | | macromolecules | |
| AAA36383 | nucleobindin 1 | promotes production of DNA-specific | 5.05* |
| | | antibodies | |
| NP_776998 | peptidoglycan recognition | innate immunity pattern recognition | 9.38* |
| | protein | molecule | |
| XP_611685 | S100 calcium binding | associated with S100A8 and implicated | 6.29* |
| | protein A9 (calgranulin B) | in inflammatory response | |
| XP_593653 | S100 calcium binding | upregulation associated with | 6.7 |
| | protein A11 (calgizzarin) | proinflammatory response | |
| NP_777076 | S100 calcium binding | antimicrobial peptide ″calcitermin″ | 5.9 |
| | protein A12 (calgranulin C) | | |
| P42819 | serum amyloid A protein | involved in acute phase cytokine | 6.94 |
| | | signaling | |
| CAA67117 | xanthine dehydrogenase | superoxide anion, hydrogen oxide and | 8.0 |
| | | peroxynitrite production | |

¹Immunoglobulins typically have isoelectric points the range of 5.0-9.5. As such, not all bind to the cationic exchange resin.
*The isoelectric points of these proteins have been calculated based on the expected protein structure. (Swiss Prot/TrEMBL, www.expasy.org).

Some of the cationic fraction components (e.g lactoferrin, angiogenin) may also have minor variants,—such as variations in amino acid sequence or in degree and type of glycosylation. The present invention contemplates that these minor variants may be incorporated in the combination of the invention.

In a preferred embodiment the combination of the invention includes at least two of the following proteins: lactoferrin (Lf); lactoperoxidase (Lp); lysosomal alpha-mannosidase (LAM); immunoglobulin heavy chain (IgG); angiogenin (ANG); and/or ribonuclease4 (RNase4).

In a preferred embodiment the combination of the invention includes at least three of the following proteins: lactoferrin (Lf); lactoperoxidase (Lp); lysosomal alpha-mannosidase (LAM); immunoglobulin heavy chain (IgG); angiogenin (ANG); and/or ribonuclease4 (RNase4).

In a preferred embodiment the combination of the invention includes at least four of the following proteins: lactoferrin (Lf); lactoperoxidase (Lp); lysosomal alpha-mannosidase (LAM); immunoglobulin heavy chain (IgG); angiogenin (ANG); and/or ribonuclease4 (RNase4).

In a preferred embodiment the combination of the invention includes at least five of the following proteins: lactoferrin (Lf); lactoperoxidase (Lp); lysosomal alpha-mannosidase (LAM); immunoglobulin heavy chain (IgG); angiogenin (ANG); and/or ribonuclease4 (RNase4).

In a preferred embodiment the combination of the invention includes each of the following proteins: lactoferrin (Lf); lactoperoxidase (Lp); lysosomal alpha-mannosidase (LAM); immunoglobulin heavy chain (IgG); angiogenin (ANG); and/or ribonuclease4 (RNase4).

In a preferred embodiment the combination of the invention includes each of the following proteins: lactoferrin (Lf); lactoperoxidase (Lp); lysosomal alpha-mannosidase (LAM); sulfhydryl oxidase (QSOX); immunoglobulin heavy chain (IgG); angiogenin (ANG); and/or ribonuclease4 (RNase4).

### Medicament

In one preferred embodiment the final treatment combination, such as the composition, may be in the form of a medicament, such as a liquid, cream, gel, paste, powder, capsule, lozenge, tablet, suppository, bolus, injectable solution, spray and so forth. The medicament may be formulated for enteral or parenteral administration. The medicament may be formulated for topical administration. In preferred embodiments the combination of the invention may be used as an antiviral agent against Human Influenza; a virus of from the family Coronaviridae; and/or Herpes Simplex.

Human Influenza may be considered primarily as a respiratory infection. As such, in preferred embodiments the combination of the present invention may be formulated as a medicament for administration to the respiratory system of a subject, such as by: oral and/or nasal administration (such as inhalation). When administered by inhalation the medicament may be formulated to direct the combination to one or more particular regions of the respiratory system. For instance, where the combination is administered as a solid medicament by mouth inhalation, the solid medicament may be micronized. The micronized solid may have a defined particle size. For example, it is believed that an inhaled powdery particle that is <3 μm in diameter may primarily deposit in the respiratory regions of the peripheral lung via diffusion. An inhaled powdery particle that is between 3 and 8 μm in diameter may be deposited by sedimentation in the transitional zones of the lung. An inhaled powdery particle that is >8 μm may be deposited in the central and conducting airways (conducting zone) by inertial impaction. It is understood that different strains of human influenza may infect different parts of the respiratory system to varying degrees. On this basis it may be possible to target specific regions of the respiratory system for different viral strains by utilising differently sized particles.

Herpes Simplex, particularly Herpes Simplex-1 (HSV-1) may manifest itself in the presentation of cold sores on human subjects in particular. For the treatment of HSV-1, in preferred embodiments the combination of the present invention may be formulated as a medicament for administration to the cold sores, such as in the form of a liquid, cream, gel, paste or spray.

The combination may be formulated neat in a medicament, but typically the medicament will include at least one or more of the following: carriers, buffers, preservatives, excipients or other pharmaceutically acceptable components required to ensure the combination is in a form that is easily dispensed, used and is efficient for its intended purpose as an antiviral agent.

The medicament may be formulated for immediate release, or may be formulated for controlled release. The controlled release may provide for sustained release, delayed release, pulsatile release, or combinations thereof. Known components which could be incorporated in the medicament to achieve controlled release are well known to one skilled in the art.

The medicament may incorporate the combination of the invention at a dose that is sufficient to elicit the desired response. As used herein the expression “effective amount” refers to an amount of the combination of proteins that is effective to achieve the desired response. For example, an effective amount of the combination may be used to treat or prevent a viral infection in a subject. In such a case the desired response, such as treating the subject, will be observed through a reduction in symptoms and/or degree of infectivity, for example.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

### Methods of Administration/Contacting

As used herein, the term “preventing” refers to keeping a disease, disorder or condition from occurring in a subject that may be predisposed to the disease, disorder and/or condition but has not yet been diagnosed as having the disease, disorder and/or condition.

As used herein, the term “treating” refers to inhibiting the progression of a disease, disorder or condition in a subject already exhibiting the symptoms of the disease, disorder and/or condition, i.e., arresting the development of a disease, disorder and/or condition that has already affected the subject.

As used herein, the term “inhibiting” refers to slowing down the progression of, or preventing (a process, reaction, or function), or reducing, the viral activity.

As used herein, the term “reducing” refers to relieving the symptoms of a disease, disorder or condition in a subject already exhibiting the symptoms of the disease, disorder and/or condition, i.e., causing regression of the disease, disorder and/or condition that has already affected the subject.

As used herein, the term “subject” refers to human, primate, equine, porcine, bovine, murine, rattus, canine and feline species. Preferably the subject is a human. As used herein, the term “patient” may be used interchangeably with “subject” and “human”.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intraveneous (bolus and infusion), intracerebral, and pulmonary routes of administration.

As referred to herein the step of “contacting the virus with an effective amount of a combination of at least two proteins” refers to providing the combination in circumstances (for an appropriate time and under appropriate conditions) such that the combination directly contacts the virus, or will contact the virus such as where the combination is applied to an extracellular medium such that it will enter the cell in circumstances where the virus is intracellular.

### Methods of Manufacture and Storage

It should be appreciated that the term ‘milk’ may include any raw (or unprocessed) milk. This is taken to include raw milk which has been chilled, incubated, or stored, at either a chilled or ambient temperature.

In some embodiments the proportions of the different cationic components within the cationic fraction may be as extracted, or concentrated.

However, this should not be seen as limiting, as it may be desirable to alter or control the ratio of at least one, or a number of protein components respectively. It should be appreciated that any such alteration in the proportions of the cationic fraction components are covered by this disclosure.

In some preferred embodiments the cationic fraction may be extracted “on-farm”, during or directly after the milking process. This may be advantageous as some of the components may be lost, damaged or denatured during subsequent handling, storage, fat removal, or other processing steps.

Numerous methods may be used to prepare a combination, such as a composition, as described according to the present invention. However, cationic exchange is considered to be a preferred method of manufacture, as will discussed in further detail below.

Preferably, the method includes extracting preferred proteins from milk, including the steps of:
a) passing milk through an extraction material, and
b) eluting a cationic fraction of the bound milk components having an isoelectric point (pI) above 6.8.

In preferred embodiments the extraction material may be a cation exchange material. This may either be in the form of resin, expanded bed resin, magnetic beads, membrane or other suitable form for large scale extraction.

In preferred embodiments the cation exchange material may be any material that has sufficient mechanical strength to resist high pressures and maintain high flow rates.

In preferred embodiments the cation exchange resin may have a mean particle size in excess of 100 μm. Resins in larger bead form have been developed for use with viscous feed streams because they do not pack as closely as smaller beads therefore there are wider channels so that there is not excessive back-pressure.

Examples of suitable cation exchange resins are SP-Sepharose Big Beads, SP-Sepharose Fast Flow, SP-Toyopearl and S-Ceramic HyperD.

One example of an extraction and purification process is as follows:

Lactoferrin binds firmly to cation exchange and is the last major protein to elute in a salt gradient. Therefore a single step elution with 1M salt (80 mS-100 mS) elutes all proteins and peptides in a single fraction (cationic fraction). Elution with 80-100 mS salt following a prior 40 mS elution will yield a fraction that is primarily lactoferrin. The use of this technique to substantially retain all lactoferrin on a column may be preferred in order to first extract a protein sample that has reduced lactoferrin levels.

After lactoferrin, lactoperoxidase is the next most abundant of the cationic proteins captured by ion exchange from milk (0.03-0.075 mg/ml milk). In a salt gradient lactoperoxidase elutes from cation exchange before lactoferrin at 25-30 mS.

The growth factors EGF, IGF 1, IGF 2, TGF B1 and TGF B2 are present in milk in ng/ml quantities, and have been shown to be captured by cation exchange.

A number of other biologically active cationic peptides elute between lactoperoxidase and lactoferrin at 35-40 mS (intermediate fraction). These are likely to include angiogenin, quiescin, jacalin-like protein, and lysozyme-like proteins, such as chitinase-like protein (CLP-1) or lysosomal alpha mannosidase (LAM).

Immunoglobulins are eluted in low salt (15-20 mS).

In preferred embodiments the milk, or milk product may be passed through a membrane having cationic exchange properties, or a column packed with the cationic exchange resin or a batch reactor with suspended cationic resin, whereby the micro-components adsorb from the starting milk or product thereof onto the cationic exchange resin or membrane.

After adsorption of milk micro-components the cationic fraction is preferably extracted by elution with a salt solution.

However, this should not be seen as limiting as elution of the cationic fraction may also be via a shift in pH. This method, however, is not popular in large scale commercial processes as the high pH required to remove lactoferrin from the resin could be damaging to the lactoferrin, or in the present case any other components in the cationic fraction.

In preferred embodiments, before elution, the resin or membrane may be rinsed with a salt solution. Preferably the rinse solution may be sodium chloride or sodium bicarbonate, with conductivity between 5 and 10 mS (millisiemens/cm). This rinse step ensures that substantially all non-adsorbed milk components are rinsed off the resin or out of the membrane.

In preferred embodiments the cationic fraction may be eluted in a salt gradient between substantially 10 mS and 100 mS conductivity (0.1 to 2.0 M salt).

In some embodiments the cationic fraction may be eluted in a single fraction by passing a salt solution with conductivity between 80 and 100 mS through the column or membrane.

In preferred embodiments the elution salt may preferably be sodium chloride. However, this should not be seen as limiting as other salts including sodium acetate, sodium bicarbonate, ammonium bicarbonate, or potassium chloride may be used.

Having the cationic fraction eluted in a one-step elution provides a significant advantage. It decreases the length of extraction time thereby decreasing the possibility of bioactives being denatured. It also decreases the time, labour and cost of the extraction process. This can provide a significant advantage, especially on a large scale. Furthermore, the results suggest that the antiviral effect will be enhanced when the components of milk having a pI above 6.8 are retained as a single isolated fraction and administered together.

In preferred embodiments after initial monitoring of the protein levels in the eluted stream to determine the concentration of salt and the volumes required to elute all the protein, the typical large scale process operates on volumes rather than continuous monitoring.

In some embodiments the extraction may be undertaken in a continuous manner.

In some embodiments, the extraction may be undertaken in a batch elution.

In some embodiments the cationic fraction may be extracted by a ‘one-step’ process, by step elution.

In some embodiments the cationic fraction may be extracted using a gradient elution.

However this should not be seen as limiting as the cationic fraction may also be extracted in independent fractions and recombined to form the complete cationic fraction at a later stage.

In some embodiments the cationic fraction may undergo further treatments, by standard techniques known in the art, for example, to remove salt, or to concentrate, or to filter for sterility or to remove endotoxin. The concentrated fraction may also be lyophilised.

In preferred embodiments the cationic fraction may be concentrated to approximately 20% solids.

In the case of the cationic fraction being extracted from milk that is processed in the usual manner involving storage, transport and conversion to skim milk or whey the temperature should preferably be maintained at substantially 4-7° C. to minimize microbial growth.

In the case of the cationic fraction being extracted from whole milk the temperature should preferably be maintained at not less than 35° C. to ensure that lipids remain in a liquid state so that they can easily pass through the extraction material. And to ensure the bioactivity of the factors in the cationic fraction are maintained at or close to the endogenous state.

In some embodiments, the cationic fraction may be extracted from genetically modified animals, for example genetically modified to enhance lactoferrin production in dairy cows. One skilled in the art would realise that extraction from the milk of genetically modified animals may affect the ratio or concentrations of lactoferrin, or other components in the cationic fraction, or a whole cascade of key components.

In some embodiments, the cationic fraction may be extracted from animals that have been immunized, for example dairy cows immunized in order to create specific antibodies (immunoglobulins) in their milk. One skilled in the art would realise that extraction from the milk of immunized animals may affect the ratio or concentrations of lactoferrin, or other components in the cationic fraction such as immunoglobulins, or a whole cascade of key components.

In some embodiments the cationic fraction may be extracted from the same species of animal that the treatment substance is intended to be used on. In some embodiments the cationic fraction may be extracted from a different species of animal than the animal the treatment substance is intended to be used on. In preferred embodiments the subject of the methods of the invention will be human, and the source of the milk proteins will be non-human. For example, bovine milk will be used to extract proteins that are used in combination to treat a human suffering from a Herpes Simplex infection or Human Influenza infection.

The following examples are non-limiting and do not detract from the generality of the products and processes described herein.

### EXAMPLES

### Example 1

### Assessment of the Proteins in the Composition (i.e. the Cationic Fraction) via Mass Spectrometry

The process of producing the cationic fraction involved fractionating milk through a cation exchange resin, eluting the bound components from the resin using a salt solution, which can be either a one-step high molarity (>1M) salt or a gradient elution from a lower molarity up to over 1M, collecting the eluted components in a single fraction, and then desalting and purifying the collected fraction.

An exemplary cationic fraction was analysed for its constituent components, and the results are shown in Table 2. This shows a typical result for yield and identity of the major proteins identified in the cationic protein fraction.

This particular cationic fraction was captured from raw, whole milk.

TABLE 2
Sub-fractions from the cationic fraction, as measured by Mass
Spectrometry (MS). (Lactoperoxidase was determined via
extinction coefficient rather than MS.)

| | Total Protein | | Isoelectric |
|---|---|---|---|
| Identity from MS | (mg/ml) | % of total | point |
| lactoperoxidase | 4.2 | 8.0% | 8.3 |
| quiescin | 1.6 | 3.0% | 8.69 |
| jacalin-like protein | 1.4 | 2.7% | 8.71 |
| chitinase-like protein | 0.4 | 0.8% | 8.74 |
| angiogenin | 10.0 | 19.0% | 9 |
| Lactoferrin | 35.0 | 66.5% | 8.7 |
| TOTAL | 52.6 | 100.0% | |

Two further examples of combinations of milk proteins according to the invention are shown below in Table 3. One of these combinations (combination 1) has a low lactoferrin content, and is believed to be particularly effective against Human Influenza. The low lactoferrin content sample may be obtained by modifying the fractionation technique to separate the fraction due to lactoferrin from the other fractions.

TABLE 3

| | Combination 1 | |
|---|---|---|
| | ″low Lf″ | Combination 2 |
| Identity from MS | (% w/w) | (% w/w) |
| lactoferrin | 6.808 | 55.9 |
| lactoperoxidase | 66.227 | 22.8 |
| LAM | 0.618 | 0.3 |
| QSOX | 0.355 | 0.7 |
| lgG | 8.484 | 4.4 |
| angiogenin | 0.025 | 12.0 |
| RNase4 | 12.95 | 3.8 |
| TOTAL | 100 | 100 |

In each combination, the relative abundance (w/w %) of the named proteins is expressed as a percentage of the named protein total. This should not be seen as limiting, and the combination may include: other proteins having an isoelectric point of or above substantially 6.8 and which are extracted from milk; and/or one or more components that are not proteins having an isoelectric point of or above substantially 6.8 and which are extracted from milk. It will be understood that, where other proteins and/or other non-proteins are included, the relative abundance of the named proteins in the total composition will decrease below the values provided in table 3.

For example, in some embodiments Combination 1 and Combination 2 may be co-formulated with other unnamed proteins, and/or unidentified proteins, and/or non-protein components such that the compositions are as shown in Table 4 below and referred to as Combination 3 and Combination 4 respectively. In other embodiments Combination 1 and Combination 2 may be derived from a mixture of proteins co-isolated from a milk source, including other unnamed proteins, and/or unidentified proteins, such that the compositions are as shown in Table 4 below.

TABLE 4

| | Combination 3 | |
|---|---|---|
| | ″low Lf″ | Combination 4 |
| Identity from MS | (% w/w) | (% w/w) |
| lactoferrin | 4.684 | 44.9 |
| lactoperoxidase | 45.564 | 18.3 |
| LAM | 0.425 | 0.27 |
| QSOX | 0.244 | 0.53 |
| IgG | 5.837 | 3.56 |
| angiogenin | 0.017 | 9.64 |
| RNase4 | 8.911 | 3.06 |
| Other protein | Balance to 100 | Balance to 100 |
| TOTAL | 100 | 100 |

Formulations suitable for use in the present invention may include one or more components that are not proteins having an isoelectric point of or above substantially 6.8 and which are extracted from milk. Such additional components include: glucose; glucose oxidase; thiocyanate; and/or monolaurin. Each of these components, independently, may be present at above 0% but below 1% w/w, such as above 0% and below 0.9% w/w. Glucose oxidase; thiocyanate; and/or monolaurin may each independently be present at above 0% and below 1% w/w, such as above 0% and below 0.5%.

For example, other sample formulations which are suitable in the formulation include the following compositions shown in Table 4:

TABLE 4

| Combination (as | ″Cationic fraction″ | ″Activated cationic |
|---|---|---|
| a composition) | (w/w) | fraction″ (w/w) |
| Lactoferrin | 64.3% | 61.3 % |
| Lactoperoxidase | 22.8% | 26.6% |
| Other protein | 9.3% | 8.1% |
| Glucose | 0% | 0.845% |
| Glucose oxidase | 0% | 0.015% |
| Thiocyanate | 0.004% | 0.004% |
| Monolaurin | 0% | 0.25% |

Glucose oxidase can use glucose as a substrate to generate peroxide in situ. Other peroxide generating systems may include percarbonate or peracetate, which may be encapsulated or coated to control the release rates of the peroxides. These components may be considered to act as promoters, or adjuvants.

Thiocyanate is present in the “activated cationic fraction” and is an example of a substrate. Other examples of substrates include iodide or chloride, having countercations of sodium, potassium or calcium.

The innate lactoperoxidase system protects the eyes, nose, mouth and airways from invasion by harmful microbes and requires presence of the lactoperoxidase enzyme, peroxide and thiocyanate or halide.

H₂O₂ is naturally present in internal biological environments as it is a by-product of various oxidative processes. For example, neutrophils produce large amounts of free peroxy radicals (O₂⁻) of which the steady state concentration has been estimated to be in the micromolar range. (Ref. Hampton, M B, Kettle A J, Winterbourn C C. Inside the neutrophil phagosome: oxidants, myeloperoxidase, and bacterial killing. Blood 1998; 92:3007-17)

Peroxidases (such as lactoperoxidase) are present in biological secretions and catalyse H₂O₂ dependent oxidation of halides (thiocyanate, iodide, bromide, chloride) that can react with and kill microbes. (Ref. Klebanoff S J. Antimicrobial mechanisms in neutrophilic polymorphonuclear leukocytes. Semin. Hematol 1975; 12:117-42)

Thiocyanate is naturally present in lymph and blood, in the mammary, salivary and thyroid glands and their secretions, in synovial, cerebral, cervical and spinal fluids and in organs such as stomach and kidney. For example, thiocyanate levels measured in human trachea-bronchial secretions from intubated adult patients were 0.46+/−0.19 mM or 26.7+/−11 ppm (range 16-38 ppm). (Ref. Wijkstrom-Frei, C., El-Chemaly, S., Ali-Rachedi, R., Gerson, C., Cobas, M. A., Forteza, R., Salathe, M. and G. E. Conner. 2003. Lactoperoxidase and human airway host defense, Am. J. Respir. Cell Mol. Biol., 29:206-12).

### Example 2

### Antiviral Activity of Combination 3, Combination 4, and Pure Lactoferrin Against Human Influenza H1N1 A

Cytotoxicity (CC₅₀) and anti-viral activity (IC₅₀) against Influenza (INFV A) H1N1 A/Puerto Rico/8/34 of two combinations of the invention were evaluated using lactoferrin (Lf) as a positive control.

### Cytotoxicity Assay

Eight 2-fold dilutions of the two test combinations of the invention and Lf starting at 1,000 and 625 μM were used for one-hour incubation with MDCK cells seeded in 96-well plates in triplicate before fresh medium was added to the plate; incubation at 35° C.

After 3 days incubation, cells were lysed for evaluation of the ATP content using Promega's CellTiter-Glo® kit. The luciferase luminescence in relative light units (RFU) was read and 50 percent cytotoxicity concentration (CC50) was calculated using XLfit dose response model.

### CPE-Based Inhibition Assay

Eight 2-fold serial dilutions of the two test combinations of the invention and Lf starting at 1,000 and 625 μM were prepared in triplicate for one-hour incubation with MDCK cells seeded in 96-well plates.

INFV A were added to the mix TA-MDCK cells at a multiplicity of infection (MOI) 0.02 for one-hour incubation at 35° C. before fresh medium was added to the plate.

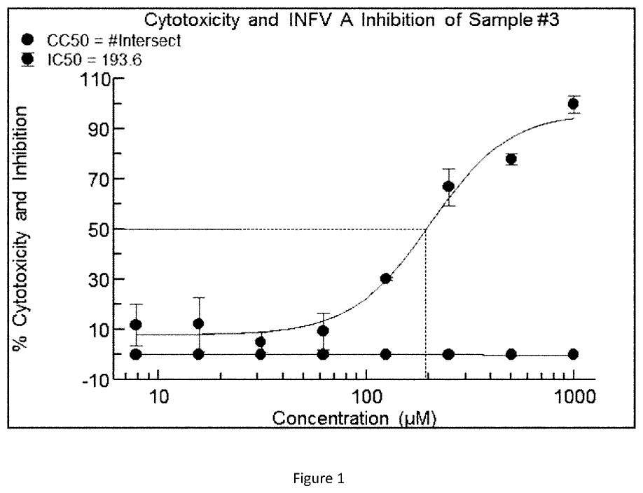

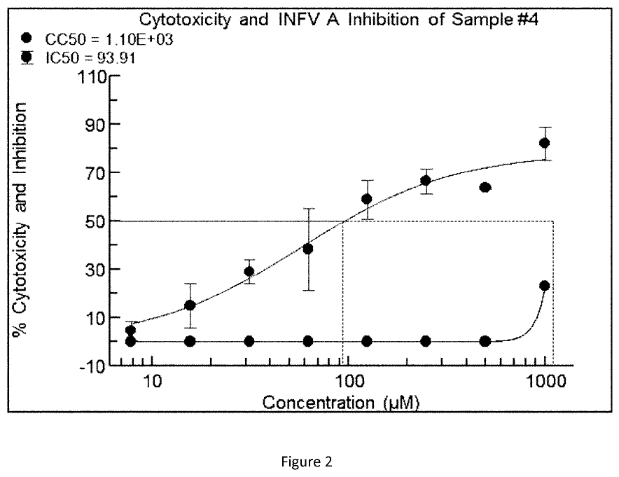

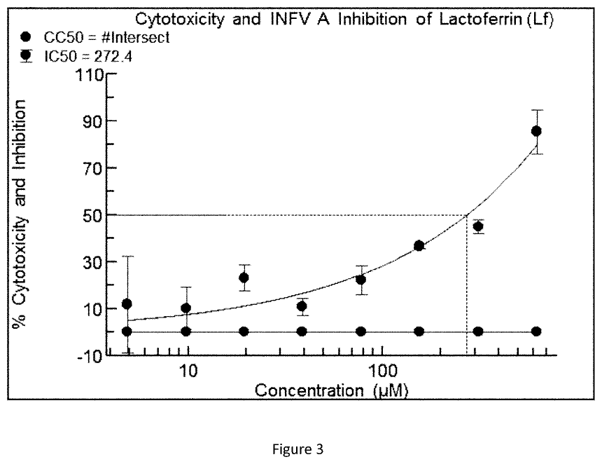

On day three post infection (3 dpi) for INFV A, MDCK cells were stained with crystal violet and optical density was read for calculation of 50 percent inhibition concentration (IC₅₀) of the two test samples and Lf using XLfit dose response model. The results are presented in FIGS. 1-3 and tabulated below in Table 5. Given that the combinations of the invention typically include protein components having potentially very different molecular weights, presentation of the IC₅₀ data in w/v is the most representative. For cytotoxicity a nominal MW of 50,000 g/mol is used for the combinations of the invention, and 80,000 g/mol for lactoferrin.

TABLE 5
Antiviral activity of two test combinations
of the invention and lactoferrin (Lf)

| | | | Stock | Start | | |
|---|---|---|---|---|---|---|
| | | MW | conc. | conc. | CC₅₀ | IC₅₀ |
| Composition | Diluent | (g/mol) | (μm) | (μM) | (M) | (mg/mL) |
| Combination | PBS | 50,000 | 10,000 | 1,000 | ND | 9.7 |
| #4 | | | | | | |
| Combination | PBS | 50,000 | 10,000 | 1,000 | 1.10E+03 | 4.7 |
| #3 | | | | | (extrapol.) | |
| Lactoferrin | PBS | 80,000 | 6,250 | 625 | ND | 21.8 |
| (96%) | | | | | | |

ND: Not detectable or #Intersect in Graphs

Zero to low cytotoxicity was observed for the each of the two test combinations and Lf.

Antiviral activity against INFV A was detected for both test combinations and Lf, although the two test combinations demonstrated enhanced activity as evident from lower IC₅₀ values. This result was surprising since the test combinations that were used contained no more than about 45% lactoferrin. Based on Lf alone, the activity of the two test combinations against INFV A might, at best, have approximately 45% of the activity of Lf—which would indicate an IC₅₀ value of about 48.4 mg/mL. Instead, the activities of combinations #3 (6.9% Lf) and #4 (44.91% Lf) were 5-68 times better than lactoferrin alone normalised for lactoferrin content which was surprising and indicates that there is an unexpected synergistic interaction between the components of the combinations of the present invention and INFV A. In particular, the low Lf combination #4 demonstrated remarkable activity demonstrating the antiviral properties of the non-Lf components in the milk protein combination according to the invention.

Without wishing to be bound by theory the inventors believe that the results from this assay indicate that the combinations of the invention are functioning to prevent docking of the viral particle to the MDCK cell surface. This mode of action is believed to be conserved for other viral particles, thus indicating a principle capable of general application to other viral targets.

### Example 3

### Antiviral Activity of Combination 3, Combination 4, Combination 5, and Pure Lactoferrin Against Herpes Simplex Type-1 (HSV-1)

Cytotoxicity (CC₅₀) and anti-viral activity (IC₅₀) against Herpes Simplex Type-1 (HSV-1) of three combinations (Combination #3, Combination #4, Combination #5) of the invention were evaluated using lactoferrin (Lf) as a positive control.

The composition of Combination 5 is shown below:

| | Combination 5 |
|---|---|
| Identity from MS | (% w/w) |
| lactoferrin | 44.9 |
| lactoperoxidase | 18.3 |
| LAM | 0.27 |
| QSOX | 0.53 |
| IgG | 3.56 |
| angiogenin | 9.64 |
| RNase4 | 3.06 |
| Glucose (less than 1%); glucose oxidase | Less than 2.5% |
| (less than 0.5%); thiocyanate (less than 0.5%); and/or | |
| monolaurin (less than 0.5%). | |
| Other protein | Balance to 100 |
| TOTAL | 100 |

### Cytotoxicity Assay

Eight 2-fold dilutions of the test combinations and lactoferrin starting at 40 and 25 μM were used for one-hour incubation with Vero cells seeded in 96-well plates in triplicate.

On 3 dpi and/or 6 dpi, cells were lysed for evaluation of the ATP content using Promega's CelltiterGlo kit. The luciferase luminescence in relative light units (RFU) was read and 50 percent cytotoxicity concentration (CC₅₀) was calculated using XLfit dose response model

### CPE-Based Inhibition Assay

Eight 2-fold serial dilutions were prepared in triplicate for one-hour incubation with Vero cells seeded in 96-well plates. HSV-1 was added to the mix test combination/lactoferrin—Vero cells at a multiplicity of infection (MOI) 0.04 for one-hour incubation at 37° C.

On day three post infection (3 dpi) for HSV-1, the Vero cells were stained with crystal violet and optical density was read for calculation of 50 percent inhibition concentration (IC₅₀) of the test combination/lactoferrin using XLfit dose response model. The results are tabulated below in Table 6. Given that the combinations of the invention typically include protein components having potentially very different molecular weights, presentation of the data in w/v is the most representative. For cytotoxicity a nominal MW of 50,000 g/mol is used for the combinations of the invention, and 80,000 g/mol for lactoferrin.

TABLE 6
Antiviral activity of two test combinations of the invention and
lactoferrin (Lf)

| | | Stock conc. | Start conc. | CC₅₀ | IC₅₀ |
|---|---|---|---|---|---|
| Composition | Diluent | (μM) | (μM) | (μM) | (mg/mL) |
| Combination #5 | PBS | 1,000 | 40 | 1.634 | 0.02 |
| Combination #4 | PBS | 1,000 | 40 | ND | 0.06 |
| Combination #3 | PBS | 1,000 | 40 | ND | 0.27 |
| Lactoferrin | PBS | 1,000 | 25 | ND | 0.03 |
| (96%) | | | | | |

ND: Not detectable or #intersect in Graphs

Zero to low cytotoxicity was observed for the each of the three test combinations and Lf.

Antiviral activity against HSV-1 was detected for each test combinations and Lf, although combination #5 demonstrated enhanced activity as evident from a lower IC₅₀ value. This result was surprising since the test combinations that were used contained no more than about 45% lactoferrin. Based on the known antiviral properties of Lf, the activity of combination #5 against HSV-1 would, at best, be expected to have approximately 45% of the activity of Lf—which would indicate an IC₅₀ value of about 0.07 mg/mL. On that basis, not only combination #5, but also combinations #4 and #3 demonstrated surprising activity. The activities of combinations #3 (6.8% Lf), #4 (44.91% Lf), and #5 (44.91% Lf) were 1.2-3.9 times better than lactoferrin alone normalised for lactoferrin content which was surprising and indicates that there is an unexpected synergistic interaction between the components of the combinations of the present invention and HSV-1.

Without wishing to be bound by theory the inventors believe that the results from this assay indicate that the combinations of the invention are functioning to prevent docking of the viral particle to the Vero cell surface. This mode of action is believed to be conserved for other viral particles, thus indicating a principle capable of general application to other viral targets.

### Example 4

### Further Examples of Combinations of at Least Two Proteins, each Protein Having an Isoelectric Point of or Above Substantially 6.8 and Which are Extracted from Milk

Tables 7, 8, 9, and 10 below provide ranges of other example combinations of the invention that may be used in the products and processes of the invention. Values are expressed in mg/g from which % of the composition can be readily calculated. LTF=lactoferrin; LPO=lactoperoxidase; LAM=lysosomal alpha mannosidase; QSOX=sulfhydryl oxidase; IgG=immunoglobulin G; JCKL=jacalin-like protein; CH3L1=chitinase-3-like protein-1; RNase4=ribonuclease4. Where a value is marked with a “-” or a zero value, that component may either: be present below the level of detection; or be absent; or may not have been tested for in the protein combination.

TABLE 7

| Component | A | B | C | D | E | F | G | H | I |
|---|---|---|---|---|---|---|---|---|---|
| LTF | 3.1 | 64.3 | 653.4 | 729.0 | 813.0 | 12.7 | 9.7 | 346.1 | 5.0 |
| LPO | 0.37 | 0.76 | 254.7 | 236.2 | 205.6 | 6.1 | 5.8 | 232.6 | 1.6 |
| LAM | 0.01 | 0.01 | 1.4 | 1.3 | 1.4 | 0.1 | 0.1 | 2.7 | 0.0 |
| QSOX | 0.00 | 0.01 | 0.65 | 0.41 | 0.18 | 0.14 | 0.13 | 6.44 | 0.05 |
| IgG | 2.7 | 4.7 | 5.0 | 5.0 | 3.7 | 3.88 | 3.29 | 34.99 | 152.30 |
| JCKL | 0.01 | 0.01 | 0.05 | 0.04 | 0.04 | — | — | — | — |
| CH3L1 | 0.01 | 0.00 | 0.00 | 0.00 | 0.00 | — | — | — | — |
| angiogenin | — | — | — | — | — | 2.8 | 2.5 | 91.5 | 0.2 |
| RNase4 | — | — | — | — | — | 1.7 | 1.5 | 60.6 | 0.3 |
| other | q.s. | q.s. | q.s. | q.s. | q.s. | q.s. | q.s. | q.s. | q.s. |
| TOTAL | 1000 | 1000 | 1000 | 1000 | 1000 | 1000 | 1000 | 1000 | 1000 |

TABLE 8

| Component | J | K | L | M | N | O | P | Q | R |
|---|---|---|---|---|---|---|---|---|---|
| LTF | 346.5 | 16.4 | 10.5 | 10.0 | 9.2 | 8.2 | 406.6 | 30.6 | 23.3 |
| LPO | 208 | 6.0 | 5.4 | 5.1 | 4.8 | 4.8 | 238.1 | 12.7 | 9.2 |
| LAM | 2.5 | 0.1 | 0.1 | 0.1 | 0.1 | 0.1 | 3.3 | 0.2 | 0.2 |
| QSOX | 4.89 | 0.08 | 0.11 | 0.10 | 0.10 | 0.10 | 5.66 | 0.15 | 0.18 |
| IgG | 26.63 | 1.95 | 1.90 | 1.76 | 1.67 | 1.65 | 34.74 | 3.93 | 3.99 |
| JCKL | — | — | — | — | — | — | — | — | — |
| CH3L1 | — | — | — | — | — | — | — | — | — |
| angiogenin | 130.61 | 2.49 | 4.66 | 5.23 | 3.98 | 2.87 | 12.7 | 0.3 | 0.2 |
| RNase4 | 76.05 | 1.04 | 1.07 | 1.37 | 2.08 | 1.76 | 7.1 | <0.2 | <0.2 |
| other | q.s. | q.s. | q.s. | q.s. | q.s. | q.s. | q.s. | q.s. | q.s. |
| TOTAL | 1000 | 1000 | 1000 | 1000 | 1000 | 1000 | 1000 | 1000 | 1000 |

TABLE 9

| Component | S | T | U | V | W | X | Y | Z | AA |
|---|---|---|---|---|---|---|---|---|---|
| LTF | 566.7 | 9.3 | 504.1 | 478.1 | 14.0 | 12.8 | 14.8 | 12.7 | 11.4 |
| LPO | 193.0 | 4.8 | 178.6 | 203.4 | 4.6 | 3.9 | 4.6 | 4.4 | 3.8 |
| LAM | 4.7 | 0.1 | 3.9 | 2.7 | 0.1 | 0.1 | 0.1 | 0.2 | 0.2 |
| QSOX | 4.13 | 0.08 | 0.17 | 0.17 | <0.06 | <0.06 | <0.06 | <0.06 | <0.06 |
| IgG | 30.42 | 1.48 | 12.9 | 8.3 | 0.50 | 0.51 | 0.54 | 0.72 | 0.65 |
| JCKL | — | — | — | — | — | — | — | — | — |
| CH3L1 | — | — | — | — | — | — | — | — | — |
| angiogenin | 41.0 | 0.4 | 23.2 | 52.0 | 2.0 | 1.9 | 2.2 | 0.9 | 0.6 |
| RNase4 | 30.0 | 1.2 | 9.4 | 11.5 | 1.1 | 1.3 | 2.2 | 0.5 | 0.6 |
| other | q.s. | q.s. | q.s. | q.s. | q.s. | q.s. | q.s. | q.s. | q.s. |
| TOTAL | 1000 | 1000 | 1000 | 1000 | 1000 | 1000 | 1000 | 1000 | 1000 |

TABLE 10

| Component | AB | AC | AD | AE |
|---|---|---|---|---|
| LTF | 11.9 | 12.1 | 572.7 | 675.6 |
| LPO | 3.9 | 4.3 | 171.0 | 155.8 |
| LAM | 0.1 | 0.1 | 1.5 | 1.5 |
| QSOX | <0.06 | <0.06 | 0.16 | 0.18 |
| lgG | 0.72 | 0.50 | 4.20 | 5.40 |
| JCKL | — | — | — | — |
| CH3L1 | — | — | — | — |
| angiogenin | 0.5 | 1.7 | 138.2 | 32.6 |
| RNase4 | 0.6 | 1.5 | 51.7 | 21.9 |
| other | q.s. | q.s. | q.s. | q.s. |
| TOTAL | 1000 | 1000 | 1000 | 1000 |

From these exemplary combinations, the ranges of values for each of the detected components are as follows:
LTF—3.1-813 mg/g;
LPO—0.37-254.7 mg/g;
LAM—0.01-4.7 mg/g;
QSOX—up to 6.44 mg/g;
IgG—0.50-152.30 mg/g;
JCKL—up to 0.05 mg/g;
CH3L1—up to 0.01 mg/g;
Angiogenin—up to 130.61 mg/g; and
RNase4—up to 76.05 mg/g.

In some embodiments the concentration of the protein components in the combination may each, independently, be selected from the following values (such that the total concentration of the protein components does not exceed 100% of the combination):
the LTF concentration in the combination is 0-900 mg/g (0-90% w/w); such as 0-850 mg/g (0-85% w/w).
the LPO concentration in the combination is 0-400 mg/g (0-40% w/w); such as 0-300 mg/g (0-30% w/w).
the LAM concentration in the combination is 0-10 mg/g (0-1% w/w); such as 0-6 mg/g (0-0.6% w/w).
the QSOX concentration in the combination is 0-15 mg/g (0-1.5% w/w); such as 0-10 mg/g (0-1% w/w).
the IgG concentration in the combination is 0-300 mg/g (0-30% w/w); such as 0-200 mg/g (0-20% w/w).
the JCKL concentration in the combination is 0-1 mg/g (0-0.01% w/w); such as 0-0.2 mg/g (0-0.02% w/w).
the CH3L1 concentration in the combination is 0-1 mg/g (0-0.01% w/w); such as 0-0.2 mg/g (0-0.02% w/w).
the ANG concentration in the combination is 0-300 mg/g (0-30% w/w); such as 0-150 mg/g (0-15% w/w).
the RNase4 concentration of the combination is 0-150 mg/g (0-15% w/w); such as 0-100 mg/g (0-10% w/w).

Within these exemplary combinations, the combinations may be broadly categorised as “low Lf” and “high Lf” combinations, wherein the LTF (otherwise known as Lf) component may be:
Low Lf—3.1-64.3 mg/g; and
High Lf—346.1-813 mg/g.

Accordingly, in some embodiments the Lf concentration in the combination is 1-100 mg/g (0.1-10% w/w); such as 2-70 mg/g (0.2-7% w/w). In other embodiments, the Lf concentration in the combination is 200-900 mg/g (20-90% w/w); such as 300-850 mg/g (30-85% w/w). As previously discusses, low Lf combinations may provide advantages against some viral targets, whereas high Lf combinations may provide advantages against other viral targets.

### Example 5

### Antiviral Activity of Combination 3, Combination 4, Combination 5, and Pure Lactoferrin Against SARS-CoV-2 (COVID-19)

Cytotoxicity (CC₅₀) and anti-viral activity (IC₅₀) against SARS-CoV-2 (COVID-19) of three combinations (Combination #3, Combination #4, Combination #5) of the invention is evaluated using lactoferrin (Lf) as a positive control.

### Cytotoxicity Assay

Eight 2-fold dilutions of the test combinations and lactoferrin starting at 40 and 25 μM are used for one-hour incubation with Vero cells seeded in 96-well plates in triplicate.

On 3 dpi and/or 6 dpi, cells are lysed for evaluation of the ATP content using Promega's CelltiterGlo kit. The luciferase luminescence in relative light units (RFU) is read and 50 percent cytotoxicity concentration (CC₅₀) is calculated using XLfit dose response model.

### CPE-Based Inhibition Assay

Eight 2-fold serial dilutions are prepared in triplicate for one-hour incubation with Vero cells seeded in 96-well plates.

SARS-CoV-2 (COVID-19) is added to the mix test combination/lactoferrin—Vero cells at a multiplicity of infection (MOI) 0.04 for one-hour incubation at 37° C.

On day three post infection (3 dpi) for SARS-CoV-2 (COVID-19), the Vero cells are stained with crystal violet and optical density is read for calculation of 50 percent inhibition concentration (IC₅₀) of the test combination/lactoferrin using XLfit dose response model. Given that the combinations of the invention typically include protein components having potentially very different molecular weights, presentation of the data in w/v is the most representative. For cytotoxicity a nominal MW of 50,000 g/mol is used for the combinations of the invention, and 80,000 g/mol for lactoferrin.

Antiviral activity against SARS-CoV-2 (COVID-19) is detected for each test combinations and Lf.

Without wishing to be bound by theory the inventors believe that the combinations of the invention will function to prevent docking of the viral particle to the Vero cell surface. This mode of action is believed to be conserved for other viral particles, thus indicating a principle capable of general application to other viral targets.

### Example 6

### Antiviral Activity of Combination 4, Combination 6, and Pure Lactoferrin Against Stomatitis Virus-Pseudotyped (rVSVSARS-CoV-2 S)

Cytotoxicity (CC₅₀) and anti-viral activity (IC₅₀) against Stomatitis Virus-pseudotyped (rVSVSARS-CoV-2 S) of two combinations (combination #4 and combination #6) of the invention were evaluated using lactoferrin (Lf) as a positive control.

The compositions of Combinations 4 and 6, freeze-dried and spray-dried lactoferrin used in Example 6 are shown below. Values are expressed in mg/g or mg/mL from which % of the composition can be readily calculated. LTF=lactoferrin; LPO=lactoperoxidase; LAM=lysosomal alpha mannosidase; QSOX=sulfhydryl oxidase; IgG=immunoglobulin G; JCKL=jacalin-like protein; RNase4=ribonuclease4. Where a value is marked with a “-” or a zero value, that component may either: be present below the level of detection; or be absent; or may not have been tested for in the protein combination.

TABLE 11

| | | | Freeze- | Spray- |
|---|---|---|---|---|
| Identity from | Combination | Combination | dried Lf | dried Lf |
| MS | 4 (mg/g) | 6 (mg/mL) | (mg/ml) | (mg/ml) |
| LTF | 449.1 | 359.4 | 654.7 | 644.8 |
| LPO | 183.0 | 228.4 | 0.2 | 2.7 |
| LAM | 2.7 | 1.3 | 0.4 | 0.7 |
| QSOX | 5.30 | 10.7 | 0.02 | 1.71 |
| lgG | 35.60 | 134.7 | 1.4 | 0.7 |
| JCKL | — | 0.16 | — | 0.01 |
| ANG | 96.4 | 18.3 | 1.3 | 2.3 |
| RNase4 | 30.6 | 12.9 | 1.1 | 2.7 |
| TOTAL | 802.7 | 765.8 | 659.1 | 655.6 |

### Cytotoxicity Assay and CPE-Based Inhibition Assay

Freeze dried, spray dried Lf and the two combinations were obtained. In vitro trials were set up to show the effect of the two combinations on the ability of rVSVSARS-CoV-2 S to enter cells. The pseudotyped virus was used as a model for SARS-CoV-2 due to health and safety considerations. The antiCOVID 19 effect of the two combinations was compared with Lf. The trials were conducted independently by Integrated BioTheraputics Inc. (IBT, Rockville, USA).

A tissue culture based assay was employed using Vero cells. Cytotoxicity of the two combinations and lactoferrin was measured. For the inhibitory concentration assay rVSV-SARS-CoV-2 S particles were exposed to the two combinations and Lf at a range of concentrations. Following exposure, the rVSVSARS-CoV-2 S particles were added to plates containing Vero cell cultures and incubated for 24 hours. The degree infection was determined by measuring Firefly Luciferase activity, detected using the Bright-Glo™ Assay System kit (Promega), and the IC₅₀ was calculated using XLfit dose response model.

Low levels of cytotoxicity were detected only when the sample concentration was high. Cytotoxicity did not interfere with determining the IC₅₅₀.

Given that the combinations of the invention typically include protein components having potentially very different molecular weights, presentation of the data in w/v is the most representative. A nominal MW of 50,000 g/mol is used for the combinations of the invention, and 80,000 g/mol for lactoferrin.

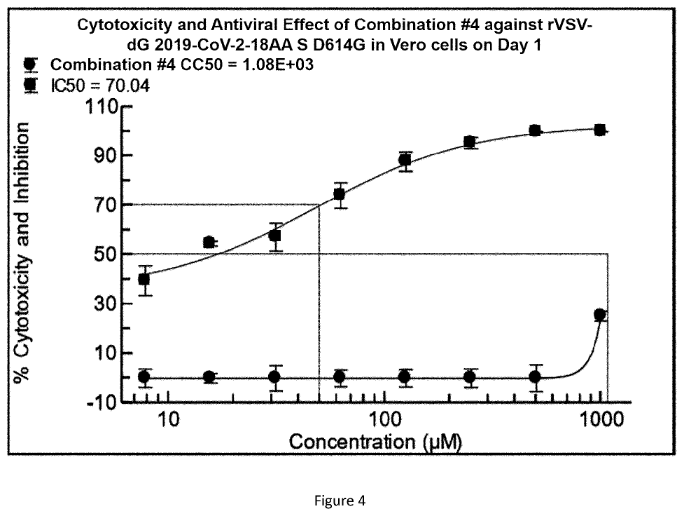

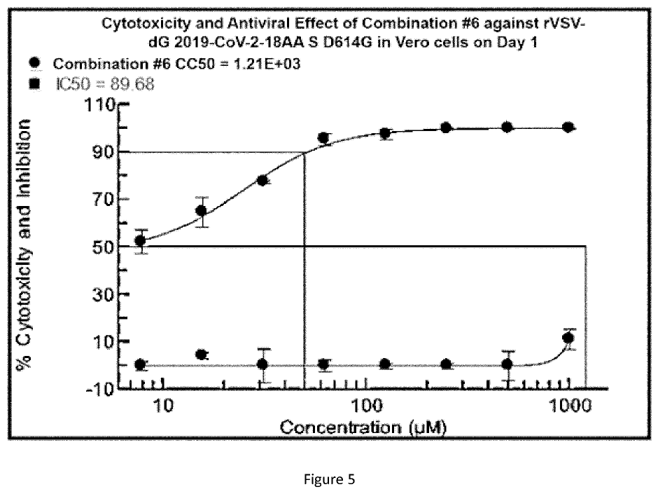

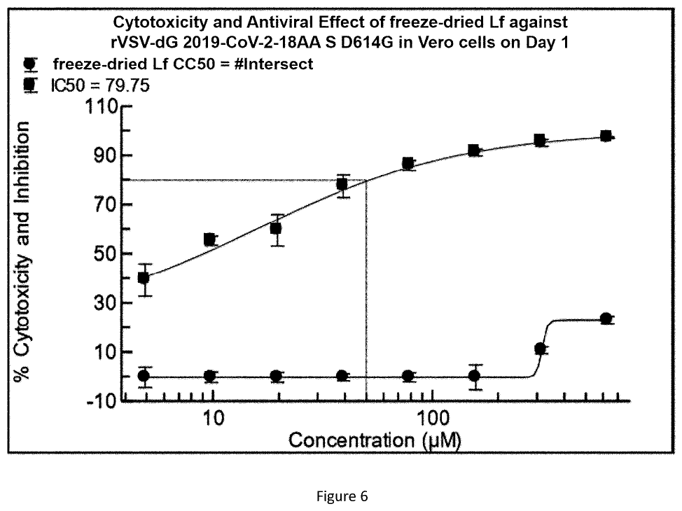

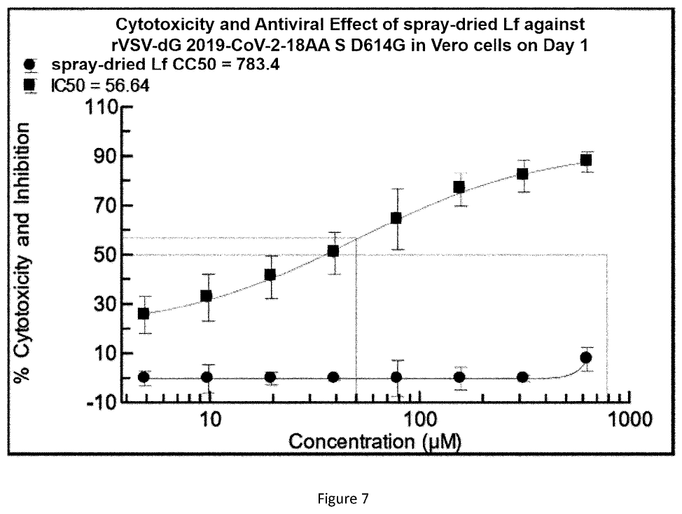

FIGS. 4-7 show the cytotoxicity and inhibition of rVSV-SARS-CoV-2 S by the two combinations and lactoferrin samples. The two combinations had IC₅₀ values of 3.5 mg/ml and 4 mg/ml. The freeze dried lactoferrin sample had an IC₅₀ value of 6.4 mg/ml and the spray dried lactoferrin sample had an IC₅₀ value of 4.5 mg/ml.

### Discussion

In vitro anti-SARS-CoV-2 studies

These in vitro trials showed that combinations of the invention inhibited the ability of rVSV-SARS-CoV-2 S to infect host cells. The results compared favorably with those from a freeze dried and spray dried lactoferrin samples, the IC₅₀'s of the combinations of the invention occurring at the same or lower concentrations.

The pseudovirus used in this assay models the interaction of the SARS-CoV-2 surface proteins with host cells. Consideration should be given to using the combinations of the invention (which may contain lactoferrin and a range of other proteins) for their potential to modulate the immune system to react appropriately to SARS-CoV-2.

### Conclusion

These results indicate that combinations of the invention have antiCOVID properties as good as, or possibly better than lactoferrin. This antiCOVID activity make the combinations of the invention an ideal ingredient for nutritional and dietary supplements, as well as topical products such as drops, creams or sprays.

Without wishing to be bound by theory the inventors believe that the combinations of the invention will function to prevent docking of the viral particle to the Vero cell surface. This mode of action is believed to be conserved for other viral particles, thus indicating a principle capable of general application to other viral targets.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. A method of preventing, inhibiting, or reducing the viral activity of a virus on or in a cell or on or in a subject, the method including the step of contacting the virus or the cell with an effective amount of a combination of at least two proteins, each protein having an isoelectric point of or above substantially 6.8 and which are extracted from milk.

2. (canceled)

3. A method of treating or preventing a viral infection in a subject, the method including the step of administering to the subject an effective amount of a combination of at least two proteins, each protein having an isoelectric point of or above substantially 6.8 and which are extracted from milk.

4-6. (canceled)

7. The method of claim 1 wherein the virus is selected from:
i. a strain from the family Coronaviridae;
ii. a Human Influenza virus; and/or
iii. a Herpes Simplex virus.

8. The method according to claim 7 wherein the strain from the family Coronaviridae is a strain from the subfamily Orthocoronavirinae.

9. The method according to claim 7 wherein the strain from the family Coronaviridae is a SARS virus.

10. The method according to claim 9 wherein the strain from the family Coronaviridae is COVID-19.

11. The method according to claim 7 wherein the Human Influenza virus is a Human Influenza A virus.

12. The method according to claim 7 wherein the Human Influenza virus is Human Influenza A H1N1.

13. The method according to claim 7 wherein the Herpes Simplex virus is HSV-1 or HSV-2.

14. The method according to claim 1 wherein where the combination includes lactoferrin the lactoferrin content of the combination is less than 40% w/w.

15. The method according to claim 1 wherein where the combination includes lactoferrin the lactoferrin content of the combination is less than 10% w/w.

16. A combination of:
(i) a combination of at least two proteins, each protein having an isoelectric point of or above substantially 6.8 and which are extracted from milk; and
(ii) at least one antiviral agent.

17. The combination according to claim 16 wherein the antiviral agent is selected from: Abacavir (HIV); Acyclovir (Aciclovir) (Herpes Simplex); Adefovir (Hepatitis B); Amantadine (Influenza); Ampligen (Avian Influenza); Amprenavir (Agenerase) (HIV); Umifenovir (Arbidol) (Influenza); Atazanavir (HIV); Atripla (HIV); Baloxavir marboxil (Xofluza) (Influenza A, Influenza B); Biktarvy (HIV); Boceprevir (Hepatitis C); Bulevirtide (Hepatitis D and Hepatitis B); Cidofovir (AIDS); Cobicistat (Tybost) (HIV); Combivir (HIV); Daclatasvir (Daklinza) (Hepatitis C); Darunavir (HIV); Delavirdine (Hepatitis C); Descovy (Hepatitis B); Didanosine (HIV); Docosanol (Herpes Simplex); Dolutegravir (HIV); Doravirine (Pifeltro) (HIV); Edoxudine (Herpes Simplex); Efavirenz (HIV); Elvitegravir (HIV); Emtricitabine (HIV); Enfuvirtide (HIV); Entecavir (HIV); Etravirine (Intelence) (HIV); Famciclovir (Herpes Zoster); Fomivirsen (AIDS); Fosamprenavir (HIV); Foscarnet (Herpes); Ganciclovir (Cytovene) (Cytomegalovirus (CMV)); Ibacitabine (Herpes labialis); Ibalizumab (Trogarzo) (HIV); Idoxuridine (Herpes); Imiquimod (Genital wart); Imunovir (Herpes Simplex); Indinavir (HIV); Lamivudine (HIV); Letermovir (Prevymis) (Cytomegalovirus (CMV)); Lopinavir (HIV); Loviride (HIV); Maraviroc (HIV); Methisazone (Smallpox); Moroxydine (Influenza); Nelfinavir (HIV); Nevirapine (HIV); Nexavir (formerly Kutapressin) (Herpes Zoster); Nitazoxanide (Broad-spectrum antiviral); Norvir (HIV); Oseltamivir (Tamiflu) (Influenza); Penciclovir (Herpes); Peramivir (Influenza); Penciclovir (Herpes); Peramivir (Rapivab) (Influenza); Pleconaril (Picornavirus); Podophyllotoxin (Genital wart); Raltegravir (HIV); Remdesivir (COVID-19); Ribavirin (Hepatitis C); Rilpivirine (HIV); Rimantadine (Influenza A); Ritonavir (HIV); Saquinavir (HIV); Simeprevir (Olysio) (Hepatitis C); Sofosbuvir (Hepatitis C); Stavudine (HIV); Taribavirin (Viramidine) (Hepatitis Syndromes in which Ribavirin is active); Telaprevir (Hepatitis C); Telbivudine (Tyzeka) (Hepatitis B); Tenofovir alafenamide (Hepatitis B); Tenofovir disoproxil (Hepatitis B, HIV); Tipranavir (HIV); Trifluridine (Eye related Herpes); Trizivir (HIV); Tromantadine (Herpes Simplex); Truvada (HIV); Umifenovir (Influenza); Valaciclovir (Valtrex) (Herpes Simplex, Herpes Zoster); Valganciclovir (Valcyte) (HIV); Vicriviroc (HIV-1); Vidarabine (Herpes Simplex, Varicella Zoster); Zalcitabine (HIV); Zanamivir (Relenza) (Influenza A, Influenza B); and Zidovudine (HIV).

18. A combination of at least two proteins, each protein having an isoelectric point of or above substantially 6.8 and which are extracted from milk, wherein where the combination includes lactoferrin the lactoferrin content of the combination is less than 40% w/w.

19. The combination according to claim 18 wherein where the combination includes lactoferrin the lactoferrin content of the combination of the at least two proteins is less than 10% w/w.
